(19) United States
(12) Patent Application Publication
(10) Pub. No.: US 2008/0119545 A1
(43) Pub. Date: May 22, 2008

(54) Method for preventing and treating avian influenza in human

(76) Inventors: Charles Hensley, Cypress, CA (US); Sung Pyo, Cypress, CA (US)

Correspondence Address: Raymond Y. Chan, Suite 128, 108 N. Ynez Avenue, Monterey Park, CA, 91754, US

(21) Appl. No.: 11/903,500

(22) Filed: Sep. 20, 2007

Related U.S. Application Data
(60) Provisional application No. 60/846,216, filed on Sep. 21, 2006.

(51) Int. Cl. A61K 31/352; A61K 31/192; A61P 31/16

(52) U.S. Cl. 514/456; 514/568

(57) ABSTRACT

A method and composition for preventing and treating Avian Influenza in Humans utilizes an effective quantity of polyphenolic(s) and/or its derivatives in combination with a carrier. The anti-avian influenza ingredient having a composition selected from the group consisting of theaflavin, theaflavin-3,3′-digallate, theaflavin-3-monogallate, theaflavin-3 gallate, theaflavin-3′-gallate, thearubigin, gallic acid, tannic acid, (−)-epigallocatechin gallate (EGCG), (−) epigalloatechin (EGC), (+)-epicatechin (EC), (−)-gallocatechin gallate (GCG), and catechin.

### CROSS REFERENCE OF RELATED APPLICATION

This is a non-provisional application of a provisional application having an application No. 60/846,216 and a filing date of Sep. 21, 2006.

### BACKGROUND OF THE PRESENT INVENTION

1. Field of Invention

This invention relates to methods and compositions for treating and preventing disease in Human. More particularly, the invention relates to a method and composition for treating and preventing Avian Influenza in Human.

2. Description of Related Arts

Avian influenza viruses pose significant threats to animal and human health and are a source of genetic diversity that permits the emergence of pandemic influenza (Webster et al. 1992). Direct avian-to-human influenza transmission was unknown before 1997 (Webby and Webster, 2003) when H5N1 virus jumped from chickens to humans in Hong Kong. Eighteen people were hospitalized, six died and three million chickens were slaughtered to contain the virus.

In 2003, highly pathogenic strains of avian influenza virus, including the H5N1 and H7N7 subtypes, again crossed from birds to humans and caused fatal disease. The year 2004 saw the largest outbreak of H5N1 avian flu in history prompting world governments and health authorities to call for emergency preparedness measures. This outbreak was an economic disaster for the poultry industry, caused loss of human life, and sounded alarm bells of an impending human influenza pandemic. With a human mortality rate of greater than 75% it has been estimated that a human pandemic involving H5N1 could result in 100 million human deaths world-wide.

Currently, there are no preventive or treatment drugs that have been shown to be effective against Avian Influenza in human.

Accordingly, it would be highly desirable to provide an improved method and composition for treating and preventing Avian Influenza.

### SUMMARY OF THE PRESENT INVENTION

It is a principal object to provide an improved method and composition for preventing and treating Avian Influenza.

Still further objects and advantages will become apparent from a consideration of the ensuing description.

These and other objectives, features, and advantages of the present invention will become apparent from the following detailed description and the appended claims.

### DETAILED DESCRIPTION OF THE PREFERRED EMBODIMENT

The present invention provides a composition and method for treating and/or preventing Avian Influenza in an individual.

The method comprises administering a treatment composition including a carrier and an anti-avian influenza ingredient comprising an appropriate combination of theaflavin, theaflavin-3,3′-digallate, theaflavin-3-monogallate, theaflavin-3 gallate, theaflavin-3′-gallate, thearubigin, gallic acid, tannic acid, (−)-epigallocatechin gallate (EGCG), (−) epigalloatechin (EGC), (−) epicatechin gallate (ECG), (+)-epicatechin (EC), (−)-gallocatechin gallate (GCG), and catechin.

The presently preferred theaflavins or derivatives are found in black tea and include, theaflavin, theaflavin-3,3′-digallate, theaflavin-3-monogallate, theaflavin-3 gallate, theaflavin-3′-gallate, thearubigin, gallic acid and tannic acid. We have demonstrated that theaflavin, theaflavin-3,3′-digallate, theaflavin-3-monogallate, theaflavin-3 gallate, theaflavin-3′-gallate, inhibits H5N1 infections in mammalian cells by blocking the attachment and entry into the cells. Our data indicate that the higher the number of gallate groups on the theaflavin backbone, the higher the potency against the virus.

The presently preferred catechins are found in green tea or green tea extract (GTE) and include (−)-epigallocatechin gallate (EGCG), (−) epigalloatechin (EGC), (−) epicatechin gallate (ECG), (+)-epicatechin (EC), (−)-gallocatechin gallate (GCG), and catechin. We have demonstrated that (−)-epigallocatechin gallate (EGCG) in particular inhibits H5N1 infections in mammalian cells by blocking the attachment and entry into the cells.

If desired, the theaflavin, theaflavin-3 gallate, theaflavin-3′-gallate, theaflavin-3,3′-digal, gallic acid, tannic acid, (−)-epigallocatechin gallate (EGCG), (−) epigalloatechin (EGC), (−) epicatechin gallate (ECG), (+)-epicatechin (EC), (−)-gallocatechin gallate (GCG), or catechin molecules an be modified by attaching different molecules, by removing a portion(s) of the molecules, or by removing a portion(s) of the molecules and incorporating a different structure for the removed portion of the molecules.

The carrier can be a liquid or solid or combination thereof, however a liquid carrier comprised of water and/or alcohol is presently preferred. The concentration of the anti-Avian Influenza ingredient can be in the range of 0.1% by weight to 90% by weight. The composition can be administered any desired number of times, but is presently preferably administered at least once a day for at least three days.

The quantity of molecules administered to a patient during a single treatment can vary as desired. A treatment program for a patient can comprise a single treatment or can comprise a plurality of treatments.

The dosage of the molecules required over time to inactivate the Avian Influenza virus can vary depending on the health of the patient and other factors.

When the molecules is administered via an IV directly into the bloodstream of a patient, about 100% bioavailability is presumed, and a dose of at least 50 mg, preferably 100 mg, and most preferably 200 mg of one or more of the molecules is preferred each time the molecules is administered. The molecules are administered at least once a day, preferably at least twice a day, and most preferably more than twice a day. Since the molecules are believed to have low toxicity, dosages of 1000 mg or more likely pose only a minimal side effect risk to a patient suffering from Avian Influenza.

When the molecules are administered by ingestion, the bioavailability is less than 100%, and a dose of at least 100 mg, preferably 200 mg, and most preferably 300 mg of one or more of the molecules. The molecules are administered at least once a day, preferably at least twice a day, and most preferably more than twice a day.

The molecules are administered in a desired carrier. The molecules are soluble in both water and alcohol at room temperature. Consequently, administering the molecules in a liquid composition comprising water and/or alcohol as a carrier is readily achieved. However, molecules, when ingested, can be administered in a liquid or solid carrier.

Accordingly, the formulas for Vira 38 products are as follows:

Vira 38
One dose is 10 ml

| | | |
|---|---|---|
| Elderberry | 4.0 g | 40% |
| Echinacea | 1.0 g | 10% |
| Epigallocatechin gallate (EGCG) | 1.0 g | 10% |
| Theaflavine (TF) | 1.0 g | 10% |
| N-Acetyl-L-Cysteine (NAC) | 1.0 g | 10% |
| Alpha Lipoic Acid (ALA) | 1.0 g | 10% |

FluStat
Per 10 ml

| | |
|---|---|
| EGCG | 2.0 g |
| TF | 1.0 g |
| NAC | 1.0 g |
| ALA | 1.0 g |

### Example 1

A composition containing 99.9% by weight water (as a carrier) and containing 0.1% by weight of the anti-Avian Influenza component is prepared by admixing water and theaflavin at room temperature.

### Example 2

A composition containing 99.9% by weight water (as a carrier) and containing 0.1% by weight of the anti-Avian Influenza component is prepared by admixing water and theaflavin-3,3′-digallate at room temperature.

### Example 3

A composition containing 99.9% by weight water (as a carrier) and containing 0.1% by weight of the anti-Avian Influenza component is prepared by admixing water and theaflavin-3-monogallate at room temperature.

### Example 4

A composition containing 99.9% by weight water (as a carrier) and containing 0.1% by weight of the anti-Avian Infuenza component is prepared by admixing water and theraflavin-3 gallate at room temperature.

### Example 5

A composition containing 99.9% by weight water (as a carrier) and 0.1% by weight of the anti-Avian Influenza component is prepared by admixing water and theaflavin-3′-gallate at room temperature.

### Example 6

A composition containing 99.9% by weight water (as a carrier) and 0.1% by weight of the anti-Avian Influenza component is prepared by admixing water and thearubigin at room temperature.

### Example 7

A composition containing 99.9% by weight water (as a carrier) and 0.1% by weight of the anti-Avian Influenza component is prepared by admixing water and gallic acid at room temperature.

### Example 8

A composition containing 99.9% by weight water (as a carrier) and 0.1% by weight of the anti-Avian Influenza component is prepared by admixing water and tannic acid at room temperature.

### Example 9

A composition containing 99.9% by weight water (as a carrier) and 0.1% by weight of the anti-Avian Influenza component is prepared by admixing water and (−)-epigallocatechin gallate (EGCG), at room temperature.

### Example 10

A composition containing 99.9% by weight water (as a carrier) and 0.1% by weight of the anti-Avian Influenza component is prepared by admixing water and (−) epigalloatechin (EGC), at room temperature.

### Example 11

A composition containing 99.9% by weight water (as a carrier) and 0.1% by weight of the anti-Avian Influenza component is prepared by admixing water and (−) epicatechin gallate (ECG), at room temperature.

### Example 12

A composition containing 99.9% by weight water (as a carrier) and 0.1% by weight of the anti-Avian Influenza component is prepared by admixing water and (+)-epicatechin (EC), at room temperature.

### Example 13

A composition containing 99.9% by weight water (as a carrier) and 0.1% by weight of the anti-Avian Influenza component is prepared by admixing water and (−)-gallocatechin gallate (GCG), at room temperature.

### Example 14

A composition containing 99.9% by weight water (as a carrier) and 0.1% by weight of the anti-Avian Influenza component is prepared by admixing water and catechin, at room temperature.

### Example 15

Example 1 is repeated, except ethanol is utilized as a carrier in place of water.

### Example 16

Example 2 is repeated, except ethanol is utilized as a carrier in place of water.

### Example 17

Example 3 is repeated, except ethanol is utilized as a carrier in place of water.

### Example 18

Example 4 is repeated, except ethanol is utilized as a carrier in place of water.

### Example 19

Example 5 is repeated, except ethanol is utilized as a carrier in place of water.

### Example 20

Example 6 is repeated, except ethanol is utilized as a carrier in place of water.

### Example 21

Example 7 is repeated, except ethanol is utilized as a carrier in place of water.

### Example 22

Example 8 is repeated, except ethanol is utilized as a carrier in place of water.

### Example 23

Example 9 is repeated, except ethanol is utilized as a carrier in place of water.

### Example 23

Example 10 is repeated, except ethanol is utilized as a carrier in place of water.

### Example 24

Example 11 is repeated, except ethanol is utilized as a carrier in place of water.

### Example 25

Example 12 is repeated, except ethanol is utilized as a carrier in place of water.

### Example 26

Example 13 is repeated, except ethanol is utilized as a carrier in place of water.

### Example 27

Example 14 is repeated, except ethanol is utilized as a carrier in place of water.

### Example 28

Examples 1 to 27 are repeated, except that the concentration of the anti-Avian

Influenza component is 5% instead of 0.1%.

### Example 29

Examples 1 to 27 are repeated, except that the concentration of the anti-Avian Influenza component is 10% instead of 0.1%.

### Example 30

Examples 1 to 27 are repeated, except that the concentration of the anti-Avian Influenza component is 50% instead of 0.1%.

### Example 31

Examples 1 to 27 are repeated, except that the concentration of the anti-Avian Influenza components is 90% instead of 0.1%.

### Example 32

Sixteen healthy adults are selected and are exposed to and directly contacted with the Avian Influenza virus such that it is highly likely each individual will develop symptoms associated with Avian Influenza. The adults are quarantined together to prevent spread of the disease. Eight of the adults comprise the control group. Members of the control group that eat normal meals three times a day, exercise once a day for an hour, and read, watch television or play games. The control group does not receive any antibiotics or other compositions to treat or prevent the onset of Avian Influenza. The remaining eight adults comprise the test group. Members of the test group, like the control group, also eat normal meals three times a day, exercise once a day for an hour, and, read, watch television, or play games. In addition, each member of the test group ingests once a day—for the next ten days after being exposed to and directly contacted with the Avian Influenza virus—a quantity of the composition of Example 1 containing 200 mg theaflavin. During the next ten days after being exposed to the Avian Influenza virus, seven of the adults in the control group develop Avian Influenza symptoms including a fever greater than 100.4 degrees F., headache, body aches, and an overall feeling of discomfort. Two of the adults in the control group develop a dry cough and have labored breathing. None of the adults in the test group develop any Avian Influenza symptoms.

### Example 33

Example 32 is repeated except that a quantity of the composition of Example 2 containing 200 mg of theaflavin-3,3′ digal is administered to each member of the test group instead of the composition of Example 1. Similar results are obtained.

### Example 34

Example 32 is repeated except that a quantity of the composition of Example 3 containing 200 mg of theaflavins is administered to each member of the test group instead of the composition of Example 1. Similar results are obtained.

### Example 35

Example 32 is repeated except that a quantity of the composition of Example 4 containing 200 mg of theaflavin-3 gallate is administered to each member of the test group instead of the composition of Example 1. Similar results are obtained.

### Example 36

Example 32 is repeated except that a quantity of the composition of Example 5 containing 200 mg of theaflavin-3′-gallate is administered to each member of the test group instead of the composition of Example 1. Similar results are obtained.

### Example 37

Example 32 is repeated except that a quantity of the composition of Example 6 containing 200 mg of thearubigin is administered to each member of the test group instead of the composition of Example 1. Similar results are obtained.

### Example 38

Example 32 is repeated except that a quantity of the composition of Example 7 containing 200 mg of gallic acid is administered to each member of the test group instead of the composition of Example 1. Similar results are obtained.

### Example 39

Example 32 is repeated except that a quantity of the composition of Example 8 containing 200 mg of tannic acid is administered to each member of the test group instead of the composition of Example 1. Similar results are obtained.

### Example 40

Example 32 is repeated except that the composition of Example 9 is utilized in placed of the composition of Example 1 to administer 200 mg of (−)-epigallocatechin gallate (EGCG) to each member of the test group. Similar results are obtained.

### Example 41

Example 32 is repeated except that the composition of Example 10 is utilized in place of the composition of Example 1 to administer 200 mg of (−) epigalloatechin (EGC) to each member of the test group. Similar results are obtained.

### Example 42

Example 32 is repeated except that the composition of Example 11 is utilized in place of the composition of Example 1 to administer 200 mg of and (−) epicatechin gallate (ECG) to each member of the test group. Similar results are obtained.

### Example 43

Example 32 is repeated except that the composition of Example 12 is utilized in place of the composition of Example 1 to administer 200 mg of and (+)-epicatechin (EC) to each member of the test group. Similar results are obtained.

### Example 44

Example 32 is repeated except that the composition of Example 13 is utilized in place of the composition of Example 1 to administer 200 mg (−)-gallocatechin gallate (GCG) of and to each member of the test group. Similar results are obtained.

### Example 45

Example 32 is repeated except that the composition of Example 14 is utilized in place of the composition of Example 1 to administer 200 mg catechin of and to each member of the test group. Similar results are obtained.

### Example 46

Examples 32 to 45 are repeated except that in each example the composition utilized to administer one or more of the anti-avain influenza ingredient to each member of the test group includes 95% by weight water and 5% by weight of the anti-avain influenza ingredient. Similar results are obtained.

### Example 47

Example 32 to 45 are repeated except that in each example the composition utilized to administer one or more of the anti-avian influenza ingredient to each member of the test group includes 50% by weight water and 50% by weight of anti-avian influenza ingredient, as the case may be. Similar results are obtained.

### Example 48

Examples 32 to 45 are repeated except that in each example the composition ingested by each adult in the test group includes only 100 mg of the particular anti-avian influenza ingredient recited in the examples. Similar results are obtained.

### Example 49

Examples 32 to 45 are repeated except that in each example the composition ingested by each adult in the test group includes 500 mg of the particular anti-avian influenza ingredient recited in the examples. Similar results are obtained.

### Example 50

Examples 32 to 45 are repeated except that the anti-avain influenza ingredient recited in each example is administered intravenously instead of orally. Similar results are obtained.

### Example 51

After the members of the control group in Example 32 begin to exhibit Avian Influenza symptoms, each member of the test group is administered once daily an amount of the composition of Example 1 sufficient to provide 200 mg of theaflavin to the test group member. The Avian Influenza symptoms abate more rapidly than normal and each test group member fully recovers from the Avian Influenza illness.

As noted above, the anti-Avian Influenza compositions of the invention can also be used to treat and prevent Avian Influenza and other influenza virus induced immunopathology (including by not limited to inflammation cause by cytokines); to treat and prevent Avian Influenza and other influenza infections in animals; and, as an agent to kill Avian Influenza virus and influenza viruses on the skin, countertops, surgical gloves, and other surfaces.

One embodiment of the invention comprises an article of manufacture consisting of an influenza treatment composition including a high concentration of theaflavin, theaflavin-3,3′-digal (TF-3), theaflavin-3-monogallate (TF-2), theaflavin-3 gallate, theaflavin-3′-gallate, thearubigin, gallic acid, tannic acid, (−)-epigallocatechin gallate (EGCG), (−) epigalloatechin (EGC), (−) epicatechin gallate (ECG), (+)-epicatechin (EC), (−)-gallocatechin gallate (GCG), or catechin namely including at least 5% by weight, preferably at least 10% by weight of the above mentioned compounds.

Another embodiment of the invention comprises a method for producing an influenza treatment composition, comprising the steps of processing tea leaves or other botanical sources to produce a concentrate including at least 5% by weight of theaflavin(s) and/or thearubigin. Processes for concentrating theaflavins(s) and thearubigin are known and are not detailed herein.

One skilled in the art will understand that the embodiment of the present invention described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A method for preventing and treating Avian Influenza in an individual, comprising a step of administering to said individual an anti-avian influenza ingredient having a composition, containing polyphenolic, selected from the group consisting of theaflavin, theaflavin-3,3′-digallate, theaflavin-3-monogallate, theaflavin-3 gallate, theaflavin-3′-gallate, thearubigin, gallic acid, tannic acid, (−)-epigallocatechin gallate (EGCG), (−) epigalloatechin (EGC), (+)-epicatechin (EC), (−)-gallocatechin gallate (GCG), and catechin.

2. The method, as recited in claim 1, wherein said anti-avian influenza ingredient includes said composition in combination with a carrier.

3. The method, as recited in claim 2, wherein said carrier is water admixing with said composition.

4. The method, as recited in claim 3, wherein said anti-avian influenza ingredient contains a range from 50% to 99.9% by weight of said carrier and a range from 50% to 0.1% by weight of said composition.

5. The method, as recited in claim 2, wherein said carrier is ethanol admixing with said composition.

6. The method, as recited in claim 5, wherein said anti-avian influenza ingredient contains a range from 50% to 99.9% by weight of said carrier and a range from 50% to 0.1% by weight of said composition.

7. The method, as recited in claim 1, wherein said polyphenolic is extracted from tealeaves.

8. The method, as recited in claim 4, wherein said polyphenolic is extracted from tealeaves.

9. The method, as recited in claim 6, wherein said polyphenolic is extracted from tealeaves.

10. The method, as recited in claim 1, wherein theaflavin (TF1), theaflavin-3 gallate (TF2a), theaflavin-3′-gallate (TF2b), theaflavin-3,3′-digallate (TF-3), thearubigin, gallic acid and tannic acid are extracted from black tea.

11. The method, as recited in claim 1, wherein (−)-epigallocatechin gallate (EGCG), (−) epigalloatechin (EGC), (−) epicatechin gallate (ECG), (+)-epicatechin (EC), (−)-gallocatechin gallate (GCG), and catechin are extracted in green tea.

12. The method, as recited in claim 1, wherein said anti-avian influenza ingredient is administered directly to human by ingestion.

13. A medicament composition for preventing and treating Avian Influenza in an individual, comprising an anti-avian influenza ingredient having a composition, containing polyphenolic, selected from the group consisting of theaflavin, theaflavin-3,3′-digallate, theaflavin-3-monogallate, theaflavin-3 gallate, theaflavin-3′-gallate, thearubigin, gallic acid, tannic acid, (−)-epigallocatechin gallate (EGCG), (−) epigalloatechin (EGC), (+)-epicatechin (EC), (−)-gallocatechin gallate (GCG), and catechin.

14. The medicament composition, as recited in claim 13, wherein said anti-avian influenza ingredient includes said composition in combination with a carrier.

15. The medicament composition, as recited in claim 14, wherein said carrier is water admixing with said composition.

16. The medicament composition, as recited in claim 15, wherein said anti-avian influenza ingredient contains a range from 50% to 99.9% by weight of said carrier and a range from 50% to 0.1% by weight of said composition.

17. The medicament composition, as recited in claim 14, wherein said carrier is ethanol admixing with said composition.

18. The medicament composition, as recited in claim 17, wherein said anti-avian influenza ingredient contains a range from 50% to 99.9% by weight of said carrier and a range from 50% to 0.1% by weight of said composition.

19. The medicament composition, as recited in claim 13, wherein said polyphenolic is extracted from tealeaves.

20. The medicament composition, as recited in claim 13, wherein said anti-avian influenza ingredient is in powered formed for being administered directly to human by ingestion.
